(19) United States
(12) Plant Patent Application Publication
(10) Pub. No.: US 2022/0248585 P1
(43) Pub. Date: Aug. 4, 2022

(54) BLUBERRY PLANT NAMED C13-051

(50) ;

(71) Applicants: Costa Berry International Pty Ltd., Ravenhall (AU); Florida Foundation Seed Producers, Inc., Marianna, FL (US)

(72) Inventors: Jessica Scalzo, Corindi Beach (AU); Paul M. Lyrene, Micanopy, FL (US)

(21) Appl. No.: 17/803,051

(22) Filed: Jan. 26, 2022

Related U.S. Application Data
(60) Provisional application No. 63/206,153, filed on Jan. 29, 2021.

(51) Int. Cl. A01H 6/36

(52) U.S. Cl. PLT/157

(57) ABSTRACT

The new blueberry plant variety ‘C13-051’ is provided. ‘C13-051’ is a commercial variety intended for the fresh market. The variety is produced from a cross of unpatented parents ‘FL09-001’ and ‘FL05-383’.

Latin name of the family, genus, and species: Family—Ericaceae. Genus—Vaccinium. Species—corymbosum hybrid.

Variety denomination: The new blueberry plant claimed is of the variety denominated ‘C13-051’.

### BACKGROUND OF THE INVENTION

The new variety ‘C13-051’ was selected from a population of seedlings derived from crossing the blueberry varieties known as ‘FL09-001’ (unpatented seed parent) and the variety known as ‘FL05-383’ (unpatented pollen parent). The cross was made in 2009 in Florida, USA and the seed was sown and grown in Corindi Beach, New South Wales, Australia. The new variety was selected in 2013 from among plants located on land at Corindi Beach and assigned the breeding code ‘C13-051’. Plants of ‘C13-051’ were propagated by cuttings for further evaluation and resulted to be uniform and stable. The new variety showed distinctive traits such as evergreen, with fruit of good flavor.

### SUMMARY OF THE INVENTION

The new variety ‘C13-051’ was originated from a cross of ‘FL09-001’ (unpatented seed parent) and the variety known as ‘FL05-383’ (unpatented pollen parent) in 2009 in Florida, USA.

The new blueberry variety resulted from seedlings produced in a controlled breeding programme. The cross was made in 2009 in Florida, USA and the seed was sown and grown on in Corindi Beach, New South Wales, Australia.

The new variety was selected in 2013 from among plants located on land at Corindi Beach and has since been named ‘C13-051’. Since then plants of ‘C13-051’ were propagated by cuttings for further evaluation and confirmed to be uniform and stable. Asexual reproduction of the new variety ‘C13-051’ by cutting propagation since 2013 at Corindi Beach, New South Wales, Australia has demonstrated that the new variety reproduces true to type plants.

The new variety was selected in 2013 as a single plant within a population of seedlings resulting from controlled cross of Vaccinium varieties. The seedling population was planted in an experimental block in the field at Corindi Beach, New South Wales, Australia and the selection of the new variety took place in the same block. Selection criteria were a combination of early to mid harvest season, low chilling requirement, medium to high vigour, non-deciduous type of plant (evergreen), large fruit size, good fruit flavor, and firm fruit. The new variety was subsequently evaluated for six years at the commercial farm at Corindi Beach, New South Wales, Australia.

The following characteristics of the new variety have been repeatedly observed and can be used to distinguish ‘C13-051’ as a new and distinct variety of Vaccinium corymbosum hybrid:
1. Non-deciduous (Evergreen)
2. Early to medium season crop
3. High yield
4. Low chilling requirement
5. Large fruit size, average 3.1 g
6. Excellent fruit flavor

The new blueberry variety ‘C13-051’ has maintained its distinguished characteristics throughout successive asexual propagation. The variety has been repeatedly asexually reproduced through softwood cuttings in New South Wales, Australia and the clones are phenotypically identical to the original plant.

### BRIEF DESCRIPTION OF THE DRAWINGS

The accompanying photographic illustration shows typical specimens in full color of the foliage and fruit of the new variety ‘C 13-051’. The colors are as nearly true as is reasonably possible in a color representation of this type.

FIG. 1 is a photograph of the new variety ‘C13-051’, showing 3 year old plants growing in a substrate system.

FIG. 2 is a photograph of the new variety ‘C13-051’, showing a typical dense fruit cluster.

FIG. 3 is a photograph of the new variety ‘C13-051’ showing the botanical parts of the plants (ripe fruit, green fruit, flowers and leaves).

FIG. 4 is a photograph showing the large fruit size of the new variety ‘C13-051’. In particular the ripe fruit on the bush.

FIG. 5 is a photograph of showing a typical calyx of fruit of ‘C13-051’.

FIG. 6 is a photograph of showing the typical pedicel scar of fruit of ‘C13-051’.

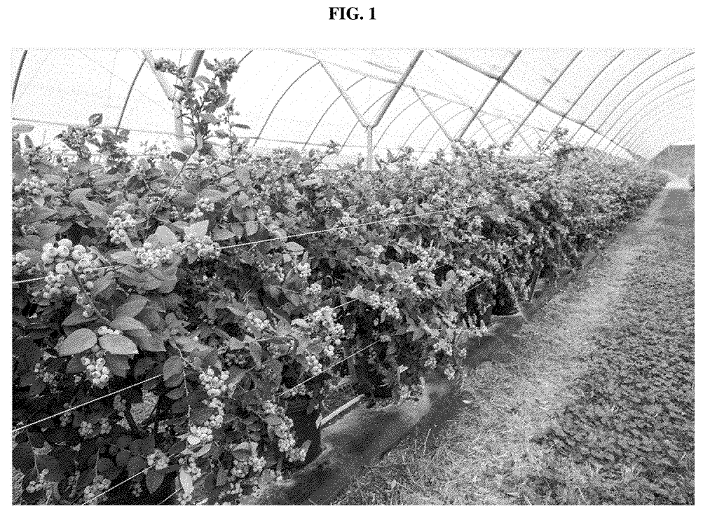

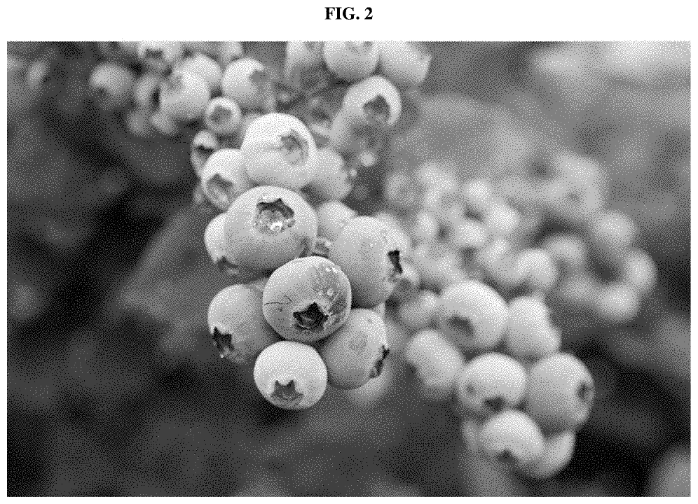

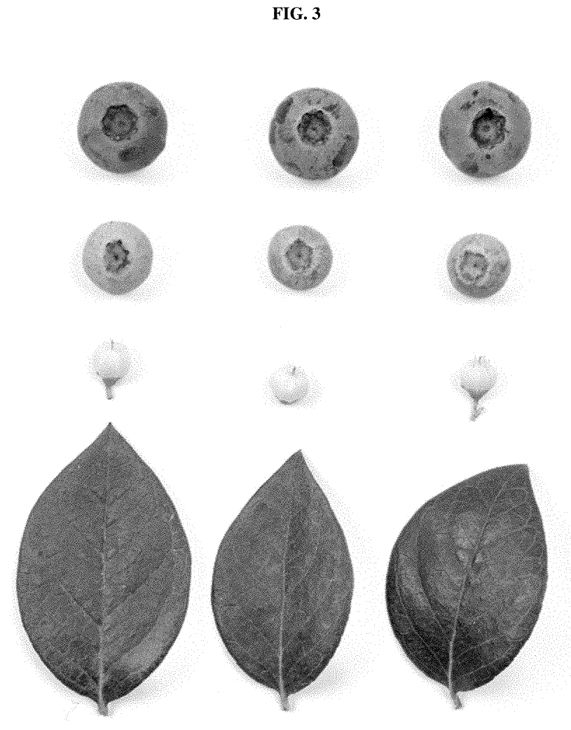

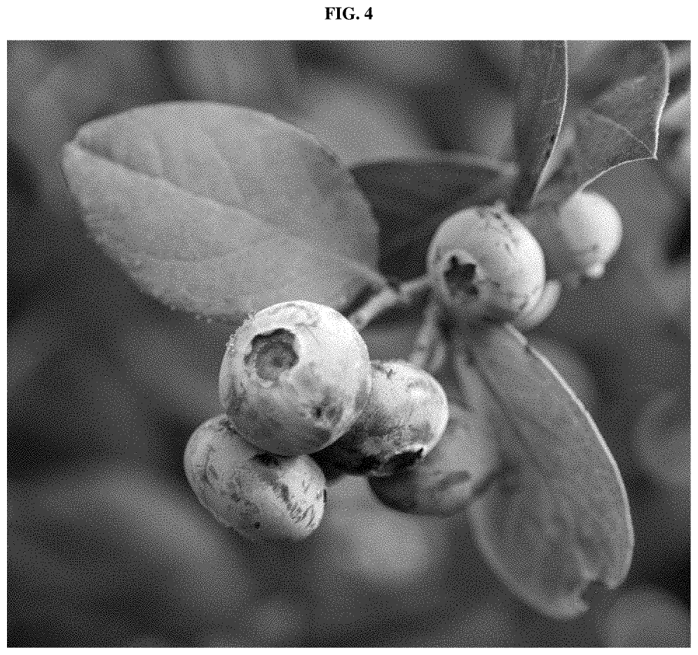

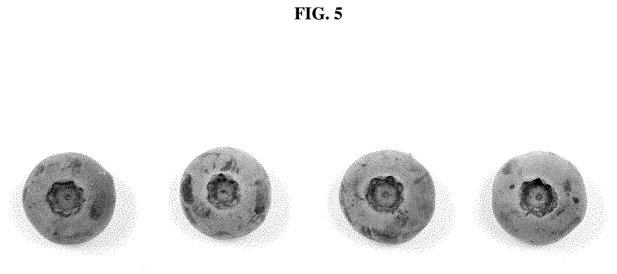

The colors in the photographs are as close as possible with the photographic and printing technology utilized. The color values cited in the detailed botanical description accurately describe the colors of the new blueberry variety.

### DETAILED BOTANICAL DESCRIPTION

The following detailed description sets forth the distinctive characteristics of ‘C13-051’. The data which defines these characteristics was collected from asexual reproductions of the original selection. Dimensions, sizes, colors, and other characteristics are approximations and averages set forth as accurately as possible. For all traits data was collected from 3 plant parts across 6 randomly selected plants. For the traits relating to fruits (e.g., fruit weight, firmness, brix, acidity) the data is an average across twenty fruits collected randomly. The plant history was taken on plants approximately 3 years of age, and the descriptions relate to plants grown in the field in Corindi Beach, New South Wales, 2456 Australia. Descriptions of fruit characteristics were made on fruit grown in Corindi Beach, New South Wales, 2456 Australia. Color designations are from the 2007 edition of The Royal Horticultural Society (“R.H.S.”).

Classification:
a. Family—Ericaceae
b. Genus—Vaccinium
c. Species—corymbosum hybrid.
d. Common Name—blueberry.

Parentage: Female Parent—unpatented selection ‘FL09-001’. Male Parent—unpatented selection ‘FL05-383’.

Market Class: fresh market.

### PLANT

General:
   Parentage.—FL09-001 x FL05-383.
   Plant height.—1.3 m.
   Plant width.—1.3 m.
   Growth habit.—Semi-upright.
   Growth.—Medium vigour.
   Productivity.—Medium to high yield.
   Cold hardiness.—Low chill.
   Cold tolerance.—Low.
   Chilling requirement.—Low, estimated between 150 and 300 hours.
   Tolerance to disease.—None assessed.
   Leafing.—Overall medium to high and plant retains leaves during the winter.
   Twigginess.—Low.

### FOLIAGE

General:
   Leaf color (top side).—Green group, similar to 139a, 137a and b.
   Leaf color (under side).—Green group, similar to 138c, 147c and 148c.
   Leaf arrangement.—Alternate.
   Leaf shape.—Elliptic.
   Leaf margins.—Entire.
   Leaf venation.—Reticulate.
   Leaf length.—Average 63.2 mm.
   Leaf width.—Average 37.4 mm.
   Leaf length/width ratio.—1.7.
   Leaf nectarines.—Absent.
   Pubescence of upper side.—Absent.
   Pubescence of lower side.—Absent.
   Cross sectional profile.—Flat.
   Longitudinal profile.—Straight.
   Attitude.—Horizontal.
Petioles:
   Length.—Average 3.9 mm.
   Width.—Average 1.8 mm.
   Color.—Yellow green 144D.

### FLOWERS

General:
   Time of beginning of flowering.—Medium.
   Flower shape.—Urceolate.
   Flower fragrance.—None perceptible.
Corolla:
   Color.—White group, similar to NN155C.
   Length.—7.03 mm.
   Width.—7.9 mm.
   Aperture width.—3.09 mm.
   Anthocyanin coloration of corolla.—Absent.
   Corolla ridges.—Present.
   Protrusion of stigma.—Present.
Inflorescence:
   Length.—
   Diameter.—
   Length of peduncle.—
   Surface texture of peduncle.—Medium.
   Color of peduncle.—Base colour is yellow-green group 145c, over colour is greyed-red group 182a .
   Length of pedicel.—8.45 mm.
   Surface texture of pedicel.—Smooth.
   Color of pedicel.—Yellow green group 145 a and c.
   Number of flowers per cluster.—11.
   Flower cluster density.—Dense.
Calyx (with sepals):
   Diameter.—6.1 mm.
Stamen:
   Length.—7.6 mm.
   Number per flower.—10.
   Filament color.—Yellow-green group 145c.
   Style.—Length — 8.1 mm.
   Color.—Yellow-green group N144b and 145b.
Pistil:
   Length.—10.1 mm.
Anther:
   Length.—3.7 mm.
   Number.—10.
   Color.—Greyed-orange group 165a.
Pollen:
   Abundance.—Abundant.
   Color.—Yellow group 10c.
   Self-compatibility.—Not yet tested.

### FRUIT

General:
   Time of fruit ripening.—Early to medium.
   Fruit development period.—On average 60 days.
   Cluster density.—Dense (8-11 berries per cluster).
   Unripe fruit color.—Similar to green group 144a.
   Ripe berry color.—Blue group 103A.
   Berry surface wax abundance.—Medium to strong.
   Berry weight.—On average 3.1 g.
   Berry height from calyx to scar.—13.3 mm.
   Berry diameter.—18.4 mm.
   Berry shape.—Oblate.
   Fruit stem scar.—Dry.
   Sweetness when ripe.—High (13.1 Brix).
   Firmness when ripe.—Medium to firm.
   Acidity when ripe.—Medium (0.55%).
   Suitability for mechanical harvesting.—Not tested.
   Uses.—Fruit to be hand harvested for fresh market.

### SEED

General:
   Seed abundance in fruit.—Abundant, on average 29 seeds per fruit.
   Seed color.—Greyed orange 166B.
   Seed length.—1.8 mm.

### COMPARISON WITH COMMERCIAL CULTIVARS

Table 1 and Table 2 below provide comparison between C13-051 and several commercial cultivars:

TABLE 1
Comparison of ‘C13-051’ with similar cultivars

| | | Corindi | | | |
|---|---|---|---|---|---|
| Characteristic | C13-051 | Verdure | C03-158 | Emerald | C97-390 |
| Soluble solid | 13.1 | 11.2 | 11.4 | 11.6 | 12.2 |
| content (%) | | | | | |
| Titratable | 0.55 | 0.45 | 0.4 | 0.5 | 0.3 |
| acidity (%) | | | | | |
| Fruit weight (g) | 3.1 | 3.1 | 2.5 | 3.0 | 2.0 |
| Fruit firmness | 173 | 251 | 240 | 238 | 219 |
| (g/mm) | | | | | |
| Plant habit | Semi- | Upright | Upright | Spreading | Semi- |
| | upright | | | | upright |

TABLE 2
Comparison of ‘C13-051’ with similar cultivars

| Characteristic | C13-051 | C99-042 | Star | Snowchaser |
|---|---|---|---|---|
| Soluble solid | 13.1 | 13.1 | 13.1 | 14.3 |
| content (%) | | | | |
| Titratable | 0.55 | 0.3 | 0.4 | 0.5 |
| acidity (%) | | | | |
| Fruit weight (g) | 3.1 | 1.9 | 2.1 | 1.7 |
| Fruit firmness | 173 | 272 | 212 | 192 |
| (g/mm) | | | | |
| Plant habit | Semi- | Semi- | Strongly | Semi- |
| | upright | upright to | upright to | upright |
| | | spreading | upright | |

1. A new and distinct variety of blueberry plant named ‘C13-051’, substantially as illustrated and described herein.
